(19) United States
(12) Patent Application Publication
(10) Pub. No.: US 2026/0174407 A1
(43) Pub. Date: Jun. 25, 2026

(54) CARDIAC PHASE SELECTION METHOD AND MEDICAL IMAGING METHOD

(71) Applicant: GE Precision Healthcare LLC, Waukesha, WI (US)

(72) Inventors: Fang Li, Beijing (CN); Darin Okerlund, Waukesha, WI (US)

(21) Appl. No.: 19/427,062

(22) Filed: Dec. 19, 2025

(30) Foreign Application Priority Data
Dec. 20, 2024 (CN) 202411895697.3

(51) Int. Cl. A61B 6/00; A61B 6/03; G06T 7/13; G06T 7/62

(52) U.S. Cl. CPC A61B 6/5288; A61B 6/03; G06T 7/13; G06T 7/62; G06T 2207/10081; G06T 2207/30048; G06T 2207/30101; G06T 2207/30168

(57) ABSTRACT

A cardiac phase selection method and an imaging method are described. The cardiac phase selection method includes: obtaining coronary image quality index data of a coronary image dataset, wherein the coronary image quality index data quantifies the medical image quality of coronary medical images of a subject under examination at a plurality of cardiac phases; selecting a first cardiac phase from the plurality of cardiac phases based on the coronary image quality index data, wherein the first cardiac phase is within a first phase range; and selecting a second cardiac phase based on the coronary image quality index data, wherein the second cardiac phase is within a second phase range different from the first phase range. In examples described herein, a plurality of cardiac phases can be selected, thereby facilitating the acquisition of medical images of the plurality of cardiac phases.

### CROSS-REFERENCE TO RELATED APPLICATIONS

This application claims priority to Chinese Application No. 202411895697.3, filed on Dec. 20, 2024, the disclosure of which is incorporated herein by reference in its entirety.

### TECHNICAL FIELD

The present specification relates to the technical field of medicine, and in particular to a cardiac phase selection method and a medical imaging method.

### BACKGROUND

Medical imaging devices are used to scan a subject under examination (such as a patient) in a non-invasive or non-destructive manner, thereby obtaining an internal structure image of a site of interest of the subject under examination to assist in diagnosis.

It should be noted that the above introduction of the background is only for the convenience of clearly and completely describing the technical solutions of the present application, and for the convenience of understanding for those skilled in the art.

### SUMMARY

During scanning of a subject under examination, motion of a site of interest affects image quality. For example, the site of interest comprises the heart, and motion of the heart may blur a coronary artery in a coronary medical image (for example, produce motion artifacts), thereby affecting a doctor's diagnosis. In the related art, an occasion with the least motion of the site of interest may be selected to perform scanning or reconstruction at the occasion with the least motion. This can reduce the influence of motion on image quality. However, in the foregoing related art, only one scanning occasion or reconstruction occasion is selected, and only a medical image of the site of interest in one motion state can be obtained. Therefore, the doctor can only make a diagnosis based on the performance of the site of interest in one motion state. The doctor relies on less information during diagnosis, and diagnostic accuracy is not high.

In view of the foregoing technical problem or other similar problems, embodiments of the present specification provide a cardiac phase selection method and a medical imaging method. In the embodiments of the present specification, a plurality of cardiac phases can be selected, so as to obtain medical images of the plurality of cardiac phases.

The embodiments of the present specification provide a cardiac phase selection method, including: obtaining coronary image quality index data of a coronary image dataset, wherein the coronary image quality index data quantifies the medical image quality of coronary medical images of a subject under examination at a plurality of cardiac phases; selecting a first cardiac phase from the plurality of cardiac phases based on the coronary image quality index data, wherein the first cardiac phase is within a first phase range; and selecting a second cardiac phase based on the coronary image quality index data, wherein the second cardiac phase is within a second phase range different from the first phase range.

The embodiments of the present specification further provide a medical imaging method, including: obtaining a coronary image dataset of a subject under examination;
determining a first cardiac phase and a second cardiac phase according to the foregoing cardiac phase selection method; and reconstructing coronary medical images of the first cardiac phase and the second cardiac phase.

The embodiments of the present specification further provide another medical imaging method, including: obtaining a coronary image quality score curve of a subject under examination, wherein the coronary image quality score curve is used to quantitatively characterize the medical image quality of a coronary medical image dataset of the subject under examination at a plurality of cardiac phases; determining a phase corresponding to a peak point in the coronary image quality score curve as a first cardiac phase, wherein a motion amplitude of the heart of the subject under examination at the first cardiac phase is less than a motion amplitude thereof at an adjacent phase, and the first cardiac phase is within one of a systolic range and a diastolic range; determining a phase corresponding to another peak point in the coronary image quality score curve as a second cardiac phase, wherein a motion amplitude of the heart of the subject under examination at the second cardiac phase is less than a motion amplitude thereof at an adjacent phase, and the second cardiac phase is within the other of the systolic range and the diastolic range; and obtaining coronary medical images at the first cardiac phase and the second cardiac phase.

The embodiments of the present specification further provide a computer program product. The computer program product comprises a computer program. The computer program, when executed by a processor, implements the foregoing cardiac phase selection method and/or the foregoing medical imaging method.

In the technical solutions of the embodiments of the present specification, a first cardiac phase and a second cardiac phase can be selected from a plurality of cardiac phases based on coronary image quality index data of the plurality of cardiac phases. The first cardiac phase and the second cardiac phase are within different phase ranges. Therefore, in the embodiments of the present specification, a plurality of cardiac phases can be selected, and the selected plurality of cardiac phases are within different phase ranges, for example, the different phase ranges comprise systole and diastole. Coronary arteries have different motion performance within different phase ranges. By obtaining coronary medical images of the first cardiac phase and the second cardiac phase, the doctor can make a diagnosis based on performance in a plurality of motion states, thereby improving diagnostic accuracy.

With reference to the following description and drawings, implementations of the embodiments of the present specification are disclosed in detail, and the manners in which the principles of the embodiments of the present specification can be employed are illustrated. It should be understood that the implementations of the present specification are not limited in scope thereby. Within the spirit and the scope of clauses of the appended claims, the implementations of the present specification comprise many changes, modifications, and equivalents.

### BRIEF DESCRIPTION OF THE DRAWINGS

The included drawings are used to provide further understanding of the embodiments of the present specification. The drawings constitute a part of the specification, and are used to illustrate the implementations of the present specification, and together with the written description, explain the technical principles of the embodiments of the present specification. Obviously, the drawings in the following description are only some embodiments of the present specification, and those of ordinary skill in the art may obtain other implementations according to the drawings without the exercise of inventive effort. In the drawings:

FIG. 1 is a schematic diagram of an architecture of a CT device in some embodiments of the present specification;

FIG. 2 is a schematic diagram of an architecture of a CT imaging system in some embodiments of the present specification;

FIG. 3 is a schematic flowchart of a cardiac phase selection method in some embodiments of the present specification;

FIG. 4 is a schematic diagram of a coronary image quality score curve in some embodiments of the present specification;

FIG. 5 is a schematic flowchart of a medical imaging method in some embodiments of the present specification;

FIG. 6 is a schematic flowchart of a medical imaging method in some embodiments of the present specification;

FIG. 7 is a schematic diagram of a structure of a cardiac phase selection apparatus in some embodiments of the present specification;

FIG. 8 is a schematic diagram of a structure of a medical imaging apparatus in some embodiments of the present specification;

FIG. 9 is a schematic diagram of a structure of a medical imaging apparatus in some embodiments of the present specification;

FIG. 10 is a schematic diagram of a structure of a medical imaging apparatus in some embodiments of the present specification; and

FIG. 11 is a schematic diagram of a ventricular volume curve in some embodiments of the present specification.

### DETAILED DESCRIPTION

The technical solutions in the embodiments of the present specification will be clearly and completely described below with reference to the drawings in the embodiments of the present specification. Obviously, the described embodiments are only some, but not all, of the embodiments of the present specification. The specific embodiments described herein are merely configured to explain the present disclosure, rather than to limit the present disclosure. All other embodiments obtained by those of ordinary skill in the art based on the described embodiments of the present disclosure fall within the scope of protection of the present disclosure. In addition, relational terms such as “first” and “second” are used merely to distinguish one entity or operation from another entity or operation, and do not necessarily require or imply that any such actual relationship or sequence exists between these entities or operations.

An electrocardiogram gating medical imaging device is used to scan a subject under examination in combination with an electrocardiogram device, to obtain an internal structure image of a site of interest of the subject under examination. The electrocardiogram device can acquire electrocardiosignals of the subject under examination. The waveform of the electrocardiosignal (for example, an R wave, which helps delineate between diastole and systole) is used to control the time to start scanning. The electrocardiogram device can send a gating signal to the electrocardiogram gating medical imaging device based on the waveform of an electrocardiogram signal. The electrocardiogram gating medical imaging device can scan the subject under examination based on the gating signal of the electrocardiogram device. In this way, the electrocardiogram gating medical imaging device can start scanning at an occasion at which cardiac motion is relatively gentle, to reduce the influence of cardiac motion on image quality. Since an electrocardiogram signal of a subject under examination needs to be acquired, imaging by an electrocardiogram gating medical imaging device takes a relatively long time. In addition, when acquiring the electrocardiogram signal, an electrode patch needs to be placed on the body of the subject under examination, which also affects the experience of the subject under examination. The electrocardiogram signal is also susceptible to electromagnetic field interference, resulting in false triggering or delayed triggering, and electrocardiogram signals of some patients may be too weak to trigger scanning.

An ECG-less cardiac gating medical imaging device can independently scan a subject under examination without combining with an electrocardiogram device, thereby obtaining an internal structure image of a site of interest of the subject under examination. The ECG-less cardiac gating medical imaging device may select an occasion with the least cardiac motion, and scanning and/or image reconstruction can be performed at the occasion with the least cardiac motion. The ECG-less cardiac gating medical imaging device includes an imaging device in which scanning and/or reconstruction is performed completely without the participation of an electrocardiogram signal, an imaging device in which a simulated electrocardiogram signal is spontaneously generated by the machine itself to participate in scanning and/or reconstruction, an imaging device in which a simulated electrocardiogram signal is generated by an external device to participate in scanning and/or reconstruction, or other types of imaging devices that can achieve the same function. Since there is no need for a gating signal of the electrocardiogram device, the ECG-less cardiac gating medical imaging device can save imaging time, improve the experience of the subject under examination, and will not affect imaging quality due to any internal or external electromagnetic field interference to which the electrocardiogram device is subjected, thereby further reducing radiation to which the subject is subjected during a radiation imaging process such as CT imaging.

In the related art, the ECG-less cardiac gating medical imaging device can select the optimal phase of the heart, and can perform scanning and/or reconstruction at the optimal phase. The heart has the least motion at the optimal phase. By performing scanning and/or reconstruction at the optimal phase, the influence of cardiac motion on image quality can be reduced. However, in the foregoing related art, only one optimal phase of the heart is selected, and thus only a medical image of the heart in one motion state can be obtained. Therefore, the doctor can only make a diagnosis based on the performance of the heart in one motion state. The doctor relies on less information during diagnosis, resulting in low diagnostic accuracy.

The embodiments of the present specification can be applied to a medical imaging system. The medical imaging system may include a medical imaging device, may include a separate computer device connected to the medical imaging device, and may further include a computer device connected to an Internet cloud. The computer device is connected via the Internet to the medical imaging device or a memory for storing medical images.

The medical imaging device may include an ECG-less cardiac gating medical imaging device, and may alternatively include an electrocardiogram gating medical imaging device. The medical imaging device is applicable to various medical imaging modalities. The medical imaging device includes, but is not limited to, a computed tomography (CT) imaging device, a positron emission tomography (PET)-CT, or any other suitable medical imaging device.

As an example, the embodiments of the present specification are described below in conjunction with an X-ray computed tomography (CT) imaging device. Those skilled in the art would appreciate that the embodiments of the present application can also be applied to other medical imaging devices.

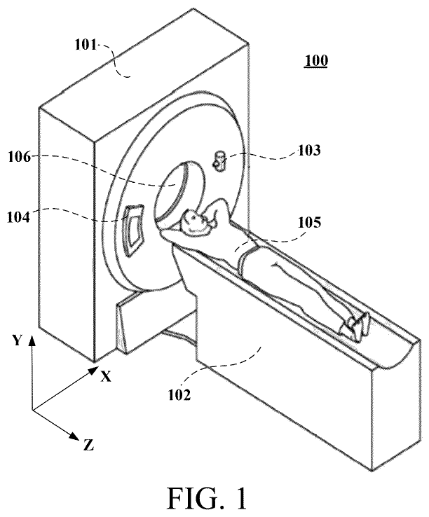

FIG. 1 is a schematic diagram of a CT device according to the embodiments of the present specification, and schematically shows a CT device 100. As shown in FIG. 1, the CT device 100 includes a scanning gantry 101 and a patient table 102. The scanning gantry 101 has a radiation source 103 (for example, an X-ray source), and the radiation source 103 projects an X-ray beam toward a detector assembly or collimator 104 on an opposite side of the scanning gantry 101. A subject under examination 105 lies flat on the patient table 102 and is moved into a scanning gantry opening 106 along with the patient table 102. A medical image (also referred to as medical image data or medical imaging data) of the subject under examination 105 may be obtained by means of scanning performed by the radiation source 103.

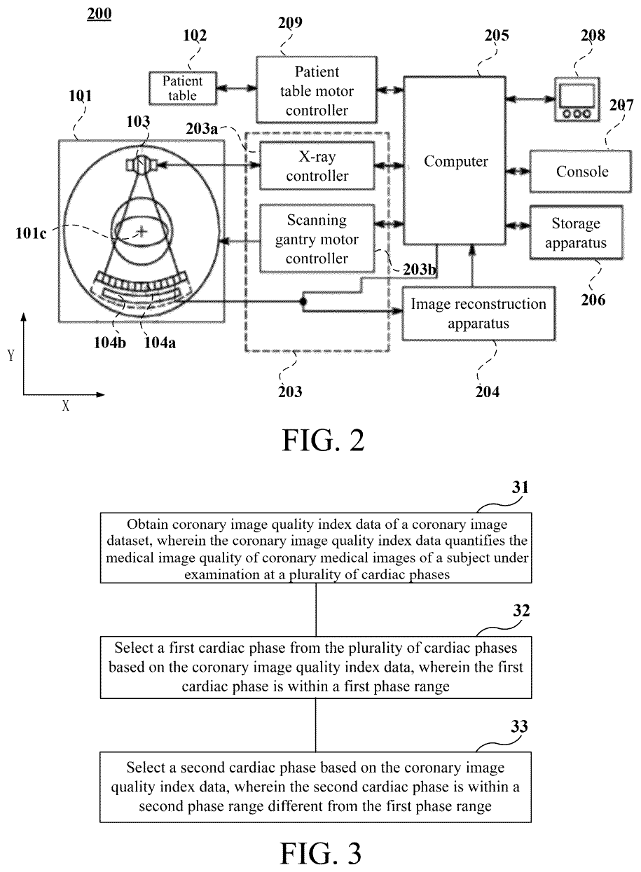

FIG. 2 is a schematic diagram of a CT imaging system according to the embodiments of the present specification, and schematically shows a block diagram of a CT imaging system 200. As shown in FIG. 2, the detector assembly 104 includes a plurality of detector units 104a and a data acquisition system (DAS) 104b. The plurality of detector units 104a sense a projected X-ray passing through the subject under examination 105.

The DAS 104b, based on the sensing of the detector units 104a, converts collected information into projection data (also referred to as scanning data) for subsequent processing. During the scanning for acquiring the X-ray projection data, the scanning gantry 101 and components mounted thereon rotate around a center of rotation 101c.

The rotation of the scanning gantry 101 and the operation of the radiation source 103 are controlled by a control mechanism 203 of the CT imaging system 200. The control mechanism 203 includes an X-ray controller 203a that provides power and a timing signal to the radiation source 103 and a scanning gantry motor controller 203b that controls the rotational speed and position of the scanning gantry 101. An image reconstruction apparatus 204 receives the projection data from the DAS 104b and executes image reconstruction. A reconstructed image is transmitted as an input to a computer 205, and the computer 205 stores the image in a mass storage apparatus 206.

The computer 205 also receives commands and scanning parameters from an operator by means of a console 207. The console 207 has an operator interface in a certain form, such as a keyboard, a mouse, a voice activated controller, or any other suitable input apparatus. An associated display 208 allows the operator to observe a reconstructed image and other data from the computer 205. The commands and parameters provided by the operator are used by the computer 205 to provide control signals and information to the DAS 104b, the X-ray controller 203a, and the scanning gantry motor controller 203b. Additionally, the computer 205 operates a patient table motor controller 209 which controls the patient table 102 so as to position the subject under examination 105 and the scanning gantry 101. In particular, the patient table 102 moves the subject under examination 105 to fully or partially pass through the scanning gantry opening 106 in FIG. 1.

In some embodiments, a reconstructed image includes a coronary medical image (also referred to as a coronary artery medical image). The computer 205 can obtain a plurality of coronary medical images inputted by the image reconstruction apparatus 204, and can determine a first cardiac phase and a second cardiac phase based on the plurality of coronary medical images. The computer 205 can send a control signal to the detector units 104a, the DAS 104b, the X-ray controller 203a, and the scanning gantry motor controller 203b. The control signal is used by the detector units 104a to sense a projected X-ray passing through the subject under examination 105 at the first phase and the second phase. The DAS 104b, based on the sensing of the detector units 104a, converts collected information into projection data. The image reconstruction apparatus 204 receives the projection data from the DAS 104b and executes image reconstruction, to obtain coronary medical images at the first phase and the second phase. The coronary medical images at the first phase and the second phase can be transmitted as input to the computer 205. The computer 205 stores the images in the mass storage apparatus 206. The doctor can make a diagnosis based on the coronary medical images at the first phase and the second phase in the storage apparatus 206.

In FIG. 1 and FIG. 2, the X direction may be, for example, a transverse or left-right direction of the scanning gantry 101 or the patient table 102; the Y direction may be, for example, an up-down direction of the scanning gantry 101 or the patient table 102; and the Z direction may be, for example, a direction in which the patient table 102 is moved in and out with respect to the scanning gantry opening 106, and the Z direction is also a front-rear direction of the scanning gantry 101 and the scanning gantry opening 106.

The device and system for obtaining medical images in the embodiments of the present specification are schematically described above, but the embodiments of the present specification are not limited thereto. The medical imaging device may be a CT device, a PET-CT, or any other suitable imaging device. The storage device may be located in the medical imaging device, in a server outside the medical imaging device, in an independent medical imaging storage system (such as a picture archiving and communication system (PACS)), and/or in a remote cloud storage system.

The embodiments of the present specification provide a cardiac phase selection method. The method can be applied to a medical imaging system. For example, the medical imaging system may be a CT imaging system or the like. The method may be independently or jointly implemented by a medical imaging device, a computer device connected to the medical imaging device, and a computer device connected to the Internet cloud.

As shown in FIG. 3, the method may include the following steps.

Step 31: Obtain coronary image quality index data of a coronary image dataset, wherein the coronary image quality index data quantifies the medical image quality of coronary medical images of a subject under examination at a plurality of cardiac phases.

Step 32: Select a first cardiac phase from the plurality of cardiac phases based on the coronary image quality index data, wherein the first cardiac phase is within a first phase range.

Step 33: Select a second cardiac phase based on the coronary image quality index data, wherein the second cardiac phase is within a second phase range different from the first phase range.

In some embodiments, cardiac motion has cyclicity. One cardiac cycle includes one complete systolic and diastolic process completed by the heart. During mid-diastole (for example, after passive ventricular filling and before atrial contraction), cardiac motion is relatively gentle. At the end of systole, mechanical contraction of the heart almost ends, and cardiac motion is also relatively gentle. Therefore, if scanning and/or reconstruction is performed during mid-diastole and/or at the end of systole, the influence of cardiac motion on image quality can be reduced.

The cardiac phase, also referred to as phase, heart phase, or period, refers to a position or occasion in a cardiac cycle. The cardiac phase may be represented by time, for example, the cardiac phase may be represented as time t, and t may be 300 milliseconds, 700 milliseconds, or the like. The cardiac phase may also be represented by a ratio, for example, the cardiac phase may be represented as a ratio r, and r may be 25%, 50%, 70%, or the like.

In some embodiments, a coronary image dataset can be obtained. The coronary image dataset includes coronary medical images (also referred to as coronary artery medical images) of the subject under examination at a plurality of cardiac phases. The plurality of cardiac phases may correspond to one cardiac cycle of the subject under examination, or may correspond to a plurality of cardiac cycles of the subject under examination. A cardiac cycle of the subject under examination may be obtained by means of estimation. For example, the cardiac cycle during scanning can be estimated by measuring the heart rate of the subject under examination before scanning. Specifically, for example, the heart rate of the subject under examination may be 60, and an estimated cardiac cycle of the subject under examination may be 1000 ms. For another example, an empirical cardiac cycle may also be used as the cardiac cycle of the subject under examination. The coronary medical images may be obtained by a medical imaging device, for example, may be obtained by an ECG-less cardiac gating medical imaging device, or may be obtained by an electrocardiogram gating medical imaging device. A coronary medical image of a cardiac phase may be obtained by performing image reconstruction on projection data (also referred to as scanning data) of the cardiac phase.

In some embodiments, coronary image quality index data of a plurality of coronary medical images in the coronary image dataset can be obtained. The coronary image dataset includes coronary medical images of the subject under examination at a plurality of cardiac phases. Therefore, the coronary image quality index data of the subject under examination at the plurality of cardiac phases can be obtained. Each piece of coronary image quality index data corresponds to one cardiac phase, and is used to quantitatively characterize the quality of the coronary medical image of the subject under examination at the cardiac phase.

The coronary image quality index data may be positively correlated with the quality of the coronary medical image, or may be negatively correlated with the quality of the coronary medical image. As an example, the coronary image quality index data may be a coronary image quality score.

For example, the coronary medical image includes three coronary vessels. The coronary image quality index data is used to quantitatively characterize the image quality of the three coronary vessels. The three coronary arteries may include the right coronary artery (RCA), the left anterior descending (LAD), and the left circumflex branch (LCX).

For example, the coronary image quality index data may include one or more of an edge strength score, a circularity score, an in-plane score, and a through-plane score of a single or a plurality of coronary arteries, or may be calculated from an edge strength score, a circularity score, an in-plane score, and a through-plane score of a single or a plurality of coronary arteries. The edge strength score is used to quantitatively characterize an edge sharpness strength and circularity at a pixel point at which a coronary artery is located. The circularity score is used to quantitatively characterize the compactness of a pixel point at which a coronary artery is located. The in-plane score is used to quantitatively characterize the image quality of a coronary vessel extending along or parallel to a plane on which a coronary image is located. The in-plane score can be calculated based on the edge strength score and the circularity score of the coronary vessel in the plane. For example, the in-plane score may be obtained by multiplying the edge strength score and the circularity score. The through-plane score is used to quantitatively characterize the image quality of a coronary vessel extending through or perpendicular to a plane on which a coronary image is located. The through-plane score can be calculated based on the edge strength score and the circularity score of the coronary vessel extending through the plane. For example, the through-plane score may be obtained by multiplying the edge strength score and the circularity score. The coronary vessel may include one or a plurality of the RCA, the LAD, or the LCX.

In some embodiments, the first cardiac phase and the second cardiac phase may be selected from a plurality of cardiac phases. The first cardiac phase is within a first phase range, and the second cardiac phase is within a second phase range. The first cardiac phase range and the second cardiac phase range are different. Therefore, in the embodiments of the present specification, a plurality of cardiac phases within different phase ranges can be obtained.

The first phase range and the second phase range correspond to different motion stages (such as systole and diastole) of the heart. The heart has different motion performance in different motion stages. The first phase range corresponds to one of systole or diastole, and the second phase range corresponds to the other of systole or diastole. Therefore, the first cardiac phase is located in one of systole or diastole, and the second cardiac phase is located in the other of systole or diastole. For example, the first cardiac phase and the second cardiac phase are used as occasions for reconstructing projection data obtained by the medical imaging device through scanning, that is, projection data of the first cardiac phase and the second cardiac phase are selected for image reconstruction, so that coronary medical images during systole or diastole can be obtained. In this way, the doctor can make a diagnosis based on the performance of a coronary artery in systole and diastole, thereby improving diagnostic accuracy.

The first phase range includes phases within a first threshold range on both sides centered on the first cardiac phase. The second phase range includes phases within the entire phase range corresponding to the coronary image dataset and outside the first phase range. The first threshold range is the product of an estimated cardiac cycle of the subject under examination and a first ratio. The first ratio is used to prevent the first cardiac phase and the second cardiac phase from being in the same motion stage of the same cardiac cycle (for example, the first cardiac phase and the second cardiac phase are in the systole of the same cardiac cycle, or in the diastole of the same cardiac cycle). The first ratio can be determined based on the ratio of one motion stage in one cardiac cycle. For example, the first ratio is half of the ratio occupied by one motion stage. The first ratio may be an empirical value, or may be determined and obtained in another manner. As an example, the first ratio is 20% or 25%. The entire phase range corresponding to the coronary image dataset may include the plurality of cardiac phases described above. For example, the entire phase range or a scanning period corresponding to the coronary image dataset covers the entire phase range or partial phase range of one estimated cardiac cycle, and a partial phase range of two adjacent estimated cardiac cycles, for example, a phase range of one half or three quarters of one estimated cardiac cycle is used for the entire phase range corresponding to the coronary image dataset.

A third phase range can be determined. The third phase range includes phases within a second threshold range on both sides centered on the first cardiac phase. The second phase is a phase within the third phase range and outside the first phase range. The second threshold range is the product of an estimated cardiac cycle of the subject under examination and a second ratio. The second ratio is used to prevent the first cardiac phase and the second cardiac phase from being in the same motion stage of different cardiac cycles (for example, the first cardiac phase is in the systole of one cardiac cycle, and the second cardiac phase is in the systole of another adjacent cardiac cycle). The second ratio can be determined based on the ratio occupied in one cardiac cycle by an interval between two adjacent and identical motion stages. The second ratio may be an empirical value, or may be determined and obtained in another manner. As an example, the second ratio is 70% or 80%.

The second phase is a phase within the third phase range and outside the first phase range. In addition, the second phase is within the second phase range, and the second phase range includes phases within the entire phase range corresponding to the coronary image dataset and outside the first phase range. Therefore, the second phase is within the intersection of the third phase range and the second phase range.

A cardiac phase within the first phase range correspondingly has coronary image quality index data. The coronary image quality index data at the first cardiac phase is a peak or one of a plurality of peaks within the first phase range. A cardiac phase within the second phase range correspondingly has coronary image quality index data. The coronary image quality index data at the second cardiac phase is a peak or one of a plurality of peaks within the second phase range. The peak is a local extremum (for example, a local maximum) of the coronary image quality index data. For example, if a piece of coronary image quality index data is greater than coronary image quality index data adjacent thereto on both sides, the piece of coronary image quality index data is a peak. Therefore, the first cardiac phase and the second cardiac phase are phases having less motion in different motion stages. The first cardiac phase and the second cardiac phase are two preferred cardiac phases in different motion stages.

In some embodiments, the coronary image quality index data is positively correlated with the quality of the coronary medical images. A cardiac phase corresponding to the maximum value of the coronary image quality index data can be selected from a plurality of cardiac phases as the first cardiac phase. Certainly, the coronary image quality index data may alternatively be negatively correlated with the quality of the coronary medical images. A cardiac phase corresponding to the minimum value of the coronary image quality index data may be selected from a plurality of cardiac phases as the first cardiac phase. Therefore, the first cardiac phase is a preferred cardiac phase within the entire phase range corresponding to the coronary image dataset. The first cardiac phase has a small motion amplitude. For example, the first cardiac phase is during mid-diastole or at the end of systole.

In some embodiments, the first phase range and the second phase range may be determined from a plurality of cardiac phases based on the first ratio. For example, the first phase range may be determined from the plurality of cardiac phases based on the first ratio, and the second phase range may be determined from the plurality of cardiac phases based on the first phase range. The third phase range may be determined from the plurality of cardiac phases based on the second ratio. The intersection of the third phase range and the second phase range may be determined. The second cardiac phase may be selected within the intersection of the third phase range and the second phase range based on the coronary image quality index data.

The coronary image quality index data is positively correlated with the quality of the coronary medical images. As an example, a cardiac phase corresponding to the maximum value of the coronary image quality index data can be selected from the intersection as the second cardiac phase. As another example, one or more other peaks other than the peak corresponding to the first cardiac phase may be obtained within the entire phase range corresponding to the coronary image dataset. A cardiac phase corresponding to the maximum value of the one or more other peaks may be selected from the intersection as the second cardiac phase. Certainly, the coronary image quality index data may alternatively be negatively correlated with the quality of the coronary medical images. As an example, a cardiac phase corresponding to the minimum value of the coronary image quality index data can be selected from the intersection as the second cardiac phase. As another example, one or more other peaks other than the peak corresponding to the first cardiac phase may be obtained within the entire phase range corresponding to the coronary image dataset. A cardiac phase corresponding to the minimum value of the one or more other peaks may be selected from the intersection as the second cardiac phase. Therefore, the second cardiac phase is another preferred cardiac phase within the entire phase range corresponding to the coronary image dataset. The second cardiac phase has a small motion amplitude. For example, the second cardiac phase is during mid-diastole or at the end of systole.

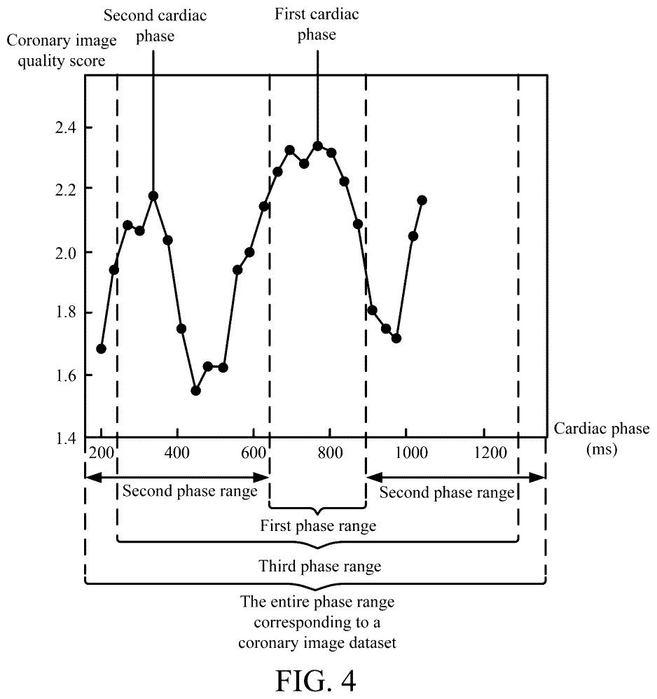

In some embodiments, a coronary image quality score curve of the subject under examination may alternatively be obtained based on the coronary image quality index data of the coronary image dataset. The coronary image quality score curve is used to quantitatively characterize the medical image quality of the coronary medical images of the subject under examination at a plurality of cardiac phases. The coronary image quality score curve includes a plurality of points. Each point represents one coronary image quality score and a cardiac phase corresponding thereto. Referring to FIG. 4, a coronary image quality score curve is schematically shown. The horizontal axis represents a cardiac phase, which is represented by means of time. The vertical axis represents a coronary image quality score.

In some embodiments, a phase corresponding to a peak point in the coronary image quality score curve may be determined as the first cardiac phase. The peak point is a local extremum (for example, a local maximum) of the coronary image quality score curve. For example, if a coronary image quality score is greater than coronary image quality scores adjacent thereto on both sides, the coronary image quality score is a peak point. The first cardiac phase corresponds to a peak point in the coronary image quality score curve, so that a motion amplitude of the heart of the subject under examination at the first cardiac phase is less than a motion amplitude at an adjacent phase. A phase corresponding to another peak point in the coronary image quality score curve may be determined as the second cardiac phase. The second cardiac phase corresponds to a peak point in the coronary image quality score curve, so that a motion amplitude of the heart of the subject under examination at the second cardiac phase is less than a motion amplitude at an adjacent phase. In some embodiments, the peak point corresponding to the first cardiac phase is the maximum value of the coronary image quality score curve.

In some embodiments, the first phase range can be determined. The first phase range includes a phase within the first threshold range on both sides centered on the first cardiac phase, and the first cardiac phase is within the first phase range. The second phase range can be determined. The second phase range includes a phase within the entire phase range corresponding to the coronary image dataset and outside the first phase range, and the second cardiac phase is within the second phase range. The third phase range can be determined, and the third phase range includes a phase within the second threshold range on both sides centered on the first cardiac phase. The second cardiac phase is a phase within the third phase range and outside the first phase range. The second cardiac phase may be selected within the intersection of the second phase range and the third phase range, and a peak point corresponding to the second cardiac phase is the maximum value in the coronary image quality score curve within the intersection.

In some embodiments, a ventricular volume dataset can also be obtained. The ventricular volume dataset includes ventricular volume data of the subject under examination at the plurality of cardiac phases described above. The ventricular volume data may be left ventricular volume data or right ventricular volume data. The first cardiac phase and the second cardiac phase can be calibrated based on the ventricular volume dataset.

A first peak and a second peak may be selected from the ventricular volume dataset. A cardiac phase corresponding to the first peak and the first cardiac phase are within the first phase range. A cardiac phase corresponding to the second peak and the second cardiac phase are within the second phase range. For example, the cardiac phase corresponding to the first peak is the first cardiac phase, and the cardiac phase corresponding to the second peak is the second cardiac phase. The first peak is one of a maximum value and a minimum value, and the second peak is the other of the maximum value and the minimum value. In another example, the cardiac phase corresponding to the first peak is different from or close to the first cardiac phase, but the distance between the two phases is small, and thus the two phases are within the same cardiac phase range, such as one of systole or diastole. The cardiac phase corresponding to the second peak is different from or close to the second cardiac phase, but the distance between the two phases is small, and thus the two phases are within the same cardiac phase range, such as the other of systole or diastole.

A motion stage corresponding to the first cardiac phase may be determined based on the first peak. A motion stage corresponding to the second cardiac phase may be determined based on the second peak. For example, under the condition that the first peak is the maximum value and the second peak is the minimum value, it can be determined that the first cardiac phase corresponds to diastole and the second cardiac phase corresponds to systole. For example, under the condition that the first peak is the minimum value and the second peak is the maximum value, it can be determined that the first cardiac phase corresponds to systole and the second cardiac phase corresponds to diastole. Therefore, motion stages corresponding to the first cardiac phase and the second cardiac phase can be determined based on the ventricular volume dataset.

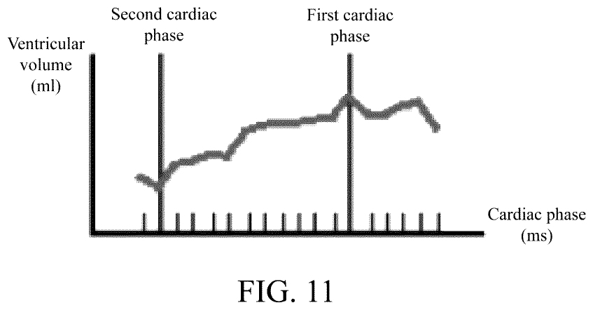

In some embodiments, a ventricular volume data curve of the subject under examination can be obtained based on the ventricular volume dataset. The ventricular volume data curve is used to represent ventricular volume data of the subject under examination at a plurality of cardiac phases. The ventricular volume data curve includes a plurality of points. Each point represents one piece of ventricular volume data and a cardiac phase corresponding thereto. Referring to FIG. 11, a ventricular volume data curve is schematically shown. The horizontal axis represents a cardiac phase. The vertical axis represents ventricular volume data.

The first peak and the second peak can be determined based on the ventricular volume data curve. A cardiac phase corresponding to the first peak is within the first phase range. A cardiac phase corresponding to the second peak is within the second phase range. The first peak is one of the maximum value and the minimum value, and the second peak is the other of the maximum value and the minimum value. As an example, the cardiac phase corresponding to the first peak is the first cardiac phase, and the cardiac phase corresponding to the second peak is the second cardiac phase. Referring to the example presented in FIG. 11, a cardiac phase corresponding to the maximum value is the first cardiac phase, and a cardiac phase corresponding to the minimum value is the second cardiac phase. Therefore, it can be determined that the first cardiac phase corresponds to diastole and the second cardiac phase corresponds to systole.

In some embodiments, coronary medical images of the subject under examination at the first cardiac phase and the second cardiac phase can also be obtained. For example, the coronary medical images of the first cardiac phase and the second cardiac phase can be reconstructed. Specifically, for example, projection data (also referred to as scanning data) of the first cardiac phase can be obtained; image reconstruction can be performed on the projection data of the first cardiac phase to obtain a coronary medical image of the first cardiac phase; projection data of the second cardiac phase can be obtained; and image reconstruction can be performed on the projection data of the second cardiac phase to obtain a coronary medical image of the second cardiac phase.

The first cardiac phase and the second cardiac phase are within different phase ranges. Coronary arteries have different motion performance within different phase ranges. By obtaining coronary medical images of the first cardiac phase and the second cardiac phase, the doctor can make a diagnosis based on performance of a coronary artery in a plurality of motion states, thereby improving diagnostic accuracy.

In the embodiments of the present specification, the first cardiac phase and the second cardiac phase can be selected from a plurality of cardiac phases based on coronary image quality index data of the plurality of cardiac phases. The first cardiac phase and the second cardiac phase are within different phase ranges. Therefore, in the embodiments of the present specification, a plurality of cardiac phases can be selected, and the plurality of cardiac phases are within different phase ranges. Coronary arteries have different motion performance within different phase ranges. By obtaining coronary medical images of the first cardiac phase and the second cardiac phase, the doctor can make a diagnosis based on performance in a plurality of motion states, thereby improving diagnostic accuracy.

The embodiments of the present specification further provide a medical imaging method. The method can be applied to a medical imaging system. For example, the medical imaging system may be a CT imaging system or the like. The method may be independently or jointly implemented by a medical imaging device, a computer device connected to the medical imaging device, and a computer device connected to an Internet cloud.

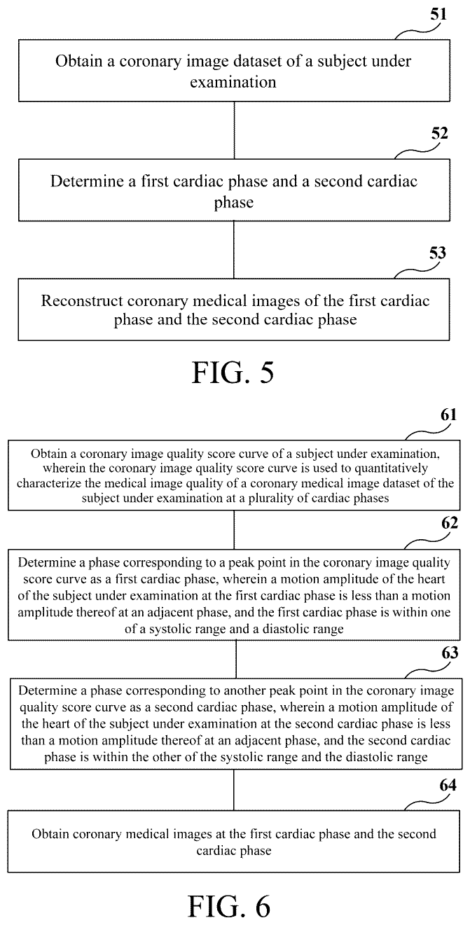

As shown in FIG. 5, the method may include the following steps.

Step 51: Obtain a coronary image dataset of a subject under examination.

Step 52: Determine a first cardiac phase and a second cardiac phase.

Step 53: Reconstruct coronary medical images of the first cardiac phase and the second cardiac phase.

In some embodiments, coronary image quality index data of a plurality of coronary medical images in the coronary image dataset can be obtained. The coronary image dataset includes coronary medical images of the subject under examination at a plurality of cardiac phases. The first cardiac phase and the second cardiac phase can be determined based on the cardiac phase selection method of the foregoing embodiments described above. Specifically, the first cardiac phase and the second cardiac phase can be selected from a plurality of cardiac phases based on the coronary image quality index data. The first cardiac phase is within a first phase range, and the second cardiac phase is within a second phase range. The first cardiac phase range and the second cardiac phase range are different. The coronary medical images of the first cardiac phase and the second cardiac phase can be reconstructed. Therefore, the doctor can make a diagnosis based on the performance of a coronary artery in a plurality of motion states, thereby improving diagnostic accuracy. The implementation and explanation of terms in the present embodiment are similar to those in the embodiments corresponding to the cardiac phase selection method. The related content is incorporated herein, and will not be repeated herein.

The embodiments of the present specification further provide another medical imaging method. The method can be applied to a medical imaging system. For example, the medical imaging system may be a CT imaging system or the like. The method may be independently or jointly implemented by a medical imaging device, a computer device connected to the medical imaging device, and a computer device connected to an Internet cloud.

As shown in FIG. 6, the method may include the following steps.

Step 61: Obtain a coronary image quality score curve of a subject under examination, wherein the coronary image quality score curve is used to quantitatively characterize the medical image quality of a coronary medical image dataset of the subject under examination at a plurality of cardiac phases.

Step 62: Determine a phase corresponding to a peak point in the coronary image quality score curve as a first cardiac phase, wherein a motion amplitude of the heart of the subject under examination at the first cardiac phase is less than a motion amplitude thereof at an adjacent phase, and the first cardiac phase is within one of a systolic range and a diastolic range.

Step 63: Determine a phase corresponding to another peak point in the coronary image quality score curve as a second cardiac phase, wherein a motion amplitude of the heart of the subject under examination at the second cardiac phase is less than a motion amplitude thereof at an adjacent phase, and the second cardiac phase is within the other of the systolic range and the diastolic range.

Step 64: Obtain coronary medical images at the first cardiac phase and the second cardiac phase.

In some embodiments, a coronary image dataset can be obtained, and the coronary image dataset includes coronary medical images of the subject under examination at a plurality of cardiac phases. The coronary image quality score curve of the subject under examination may be obtained based on the coronary image quality index data of the coronary image dataset. The coronary image quality score curve is used to quantitatively characterize the medical image quality of the coronary medical images of the subject under examination at a plurality of cardiac phases. The coronary image quality score curve includes a plurality of points. Each point represents one coronary image quality score and a cardiac phase corresponding thereto. Referring to FIG. 4, a coronary image quality score curve is schematically shown. The horizontal axis represents a cardiac phase, which is represented by means of time. The vertical axis represents a coronary image quality score. In some embodiments, the peak point corresponding to the first cardiac phase is the maximum value of the coronary image quality score curve.

In some embodiments, a first phase range can be determined. The first phase range includes phases within a first threshold range on both sides centered on the first cardiac phase. The first cardiac phase is within the first phase range. A second phase range can be determined. The second phase range includes phases within the entire phase range corresponding to the coronary image dataset and outside the first phase range. The second cardiac phase is within the second phase range. A third phase range can be determined. The third phase range includes phases within a second threshold range on both sides centered on the first cardiac phase. The second cardiac phase is a phase within the third phase range and outside the first phase range. The second cardiac phase may be selected within the intersection of the second phase range and the third phase range, and the peak point corresponding to the second cardiac phase is the maximum value in the coronary image quality score curve within the intersection.

In some embodiments, coronary medical images of the subject under examination at the first cardiac phase and the second cardiac phase can also be obtained. For example, the coronary medical images of the first cardiac phase and the second cardiac phase can be reconstructed. Specifically, for example, projection data (also referred to as scanning data) of the first cardiac phase can be obtained; image reconstruction can be performed on the projection data of the first cardiac phase to obtain a coronary medical image of the first cardiac phase; projection data of the second cardiac phase can be obtained; and image reconstruction can be performed on the projection data of the second cardiac phase to obtain a coronary medical image of the second cardiac phase.

The first cardiac phase and the second cardiac phase are within different phase ranges. Coronary arteries have different motion performance within different phase ranges. By obtaining coronary medical images of the first cardiac phase and the second cardiac phase, the doctor can make a diagnosis based on the performance of a coronary artery in a plurality of motion states, thereby improving diagnostic accuracy.

The implementation and explanation of terms in the present embodiment are similar to those in the embodiments corresponding to the cardiac phase selection method. The related content is incorporated herein, and will not be repeated herein.

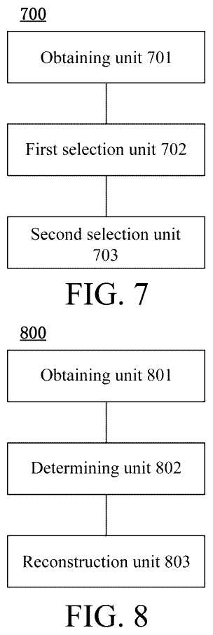

As shown in FIG. 7, some embodiments of the present specification further provide a cardiac phase selection apparatus. The cardiac phase selection apparatus can be applied to a medical imaging system. As shown in FIG. 7, a cardiac phase selection 700 may include the following units: an obtaining unit 701, configured to obtain coronary image quality index data of a coronary image dataset, wherein the coronary image quality index data quantifies the medical image quality of coronary medical images of a subject under examination at a plurality of cardiac phases; a first selection unit 702, configured to select a first cardiac phase from the plurality of cardiac phases based on the coronary image quality index data, wherein the first cardiac phase is within a first phase range; and a second selection unit 703, configured to select a second cardiac phase based on the coronary image quality index data, wherein the second cardiac phase is within a second phase range different from the first phase range.

As shown in FIG. 8, some embodiments of the present specification further provide a medical imaging apparatus. The medical imaging apparatus can be applied to a medical imaging system. As shown in FIG. 8, a medical imaging 800 may include the following units: an obtaining unit 801, configured to obtain a coronary image dataset of a subject under examination; a determining unit 802, configured to determine a first cardiac phase and a second cardiac phase; and a reconstruction unit 803, configured to reconstruct coronary medical images of the first cardiac phase and the second cardiac phase.

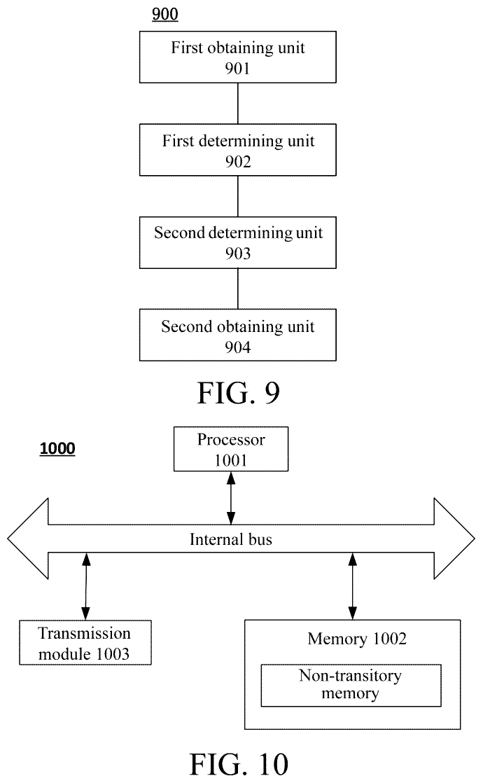

As shown in FIG. 9, some embodiments of the present specification further provide a medical imaging apparatus. The medical imaging apparatus can be applied to a medical imaging system. As shown in FIG. 9, a medical imaging 900 may include the following units: a first obtaining unit 901, configured to obtain a coronary image quality score curve of a subject under examination, wherein the coronary image quality score curve is used to quantitatively characterize the medical image quality of a coronary medical image dataset of the subject under examination at a plurality of cardiac phases; a first determining unit 902, configured to determine a phase corresponding to a peak point in the coronary image quality score curve as a first cardiac phase, wherein a motion amplitude of the heart of the subject under examination at the first cardiac phase is less than a motion amplitude thereof at an adjacent phase, and the first cardiac phase is within one of a systolic range and a diastolic range; a second determining unit 903, configured to determine a phase corresponding to another peak point in the coronary image quality score curve as a second cardiac phase, wherein a motion amplitude of the heart of the subject under examination at the second cardiac phase is less than a motion amplitude thereof at an adjacent phase, and the second cardiac phase is within the other of the systolic range and the diastolic range; and a second obtaining unit 904, configured to obtain coronary medical images at the first cardiac phase and the second cardiac phase.

It should be noted that since the implementation solution for problem-solving by the above apparatus is similar to that of the above method, for the specific implementation of the apparatus in the embodiments of the present specification, reference can be made to the implementation of the above method, and repeated details are omitted. The term “unit” used below may be a combination of software and/or hardware that implements a predetermined function. Although the apparatus described in the following embodiments is preferably implemented by software, implementation by hardware or a combination of software and hardware is also possible and conceived of.

The embodiments of the present specification further provide a medical imaging apparatus. FIG. 10 is a schematic diagram of an architecture of a medical imaging apparatus. As shown in FIG. 10, a medical imaging apparatus 1000 may include a processor 1001 and a memory 1002. The memory 1002 may include a non-transitory memory. The non-transitory memory, also referred to as a non-volatile memory, stores information that will not be lost after power is turned off. The non-transitory memory may include a magnetic storage apparatus, a flash memory, an optical disk, and the like. The memory 1002 may alternatively include a volatile memory such as a high-speed random access memory. The memory 1002 is configured to store instructions. The instructions, when executed, cause the processor 1001 to execute the method according to any one of the foregoing embodiments.

The medical imaging apparatus 1000 may further include a transmission module 1003 for a communication function. The transmission module 1003 is configured to receive or send data via a network. The specific examples of the network described above may include a wireless network provided by a communication supplier of the computer terminal. In an example, the transmission module 1003 includes a network interface controller (NIC) which may be connected to other network devices by means of a base station to communicate with the Internet. In an example, the transmission module 1003 may be a radio frequency (RF) module which is configured to communicate with the Internet in a wireless manner.

It can be understood by those of ordinary skill in the art that the structure shown in FIG. 10 is only illustrative and does not limit the structure of the medical imaging apparatus. For example, the medical imaging apparatus may also include more or fewer components than those shown in FIG. 10.

The embodiments of the present specification further provide a computer device. The computer device includes a memory, a processor, and a computer program stored in the memory and executable on the processor. When executing the computer program, the processor implements the foregoing cardiac phase selection method and/or medical imaging method.

The memory may be used to store information. The memory includes any one or a combination of: any type of RAM, any type of ROM, a flash memory device, a hard disk, an optical disk, etc. The processor may include any one or a combination of: a central processing unit (CPU), a graphics processing unit (GPU), a micro processing unit (MCU), a programmable logic device (FPGA), etc.

The embodiments of the present specification provide a computer-readable storage medium having a computer program stored thereon. When executed by a processor, the computer program implements the foregoing cardiac phase selection method and/or medical imaging method.

The computer-readable storage medium may include: apparatuses that store information using electrical energy, such as various memories, e.g., RAM, ROM, etc., apparatuses that store information using magnetic energy, such as hard disks, floppy disks, magnetic tapes, magnetic core memories, bubble memories, and U disks; and apparatuses that store information using optical means, such as CDs or DVDs. Certainly, there are other forms of readable storage media, such as quantum memories, graphene memories, etc.

The embodiments of the present specification further provide a computer program product. The computer program product includes a computer program. When executed by a processor, the computer program implements the foregoing cardiac phase selection method and/or medical imaging method.

It can be understood by those skilled in the art that the present specification may be provided as a method, a system, or a computer program product. Therefore, the present specification may be implemented in the form of a fully hardware-based embodiment, a fully software-based embodiment, or an embodiment combining software and hardware.

Furthermore, the present specification may be implemented in the form of a computer program product implemented on one or more computer-usable storage media (including, but not limited to, magnetic disk storage, CD-ROM, optical storage, etc.) having computer-usable program codes included therein.

The present specification is described with reference to flowcharts and/or block diagrams of methods, devices (systems), and computer program products according to the embodiments of the present specification. It should be understood that each flow and/or block in the flowcharts and/or block diagrams and a combination of flows and/or blocks in the flowcharts and/or block diagrams may be implemented by computer program instructions. The computer may be a personal computer, a laptop computer, a cellular telephone, a camera phone, a smart phone, a personal digital assistant, a media player, a navigation device, an email device, a gaming console, a tablet computer, a wearable device, or a combination of any of these devices.

Each functional unit in the embodiments of the present specification may be integrated into one processing unit, or each functional unit may exist alone physically, or two or more functional units may be integrated into one processing unit.

It can be understood by those skilled in the art that the description of each embodiment in the present specification has its own focus, and for a part not described in detail in a certain embodiment, reference may be made to the related description of other embodiments. In addition, it may be understood that any combination of some or all of the embodiments described in the present specification can be conceived by those skilled in the art without the exercise of any inventive effort after reading the document of the present specification, and such combinations are also within the scope of the disclosure and protection of the present specification.

Although the present specification has been described by means of embodiments, those of ordinary skill in the art will appreciate that the above embodiments are only used to facilitate understanding of the core idea of the present specification. It can be understood by those skilled in the art that many modifications and variations of the present specification are possible. It is intended that the appended claims cover such modifications and variations without departing from the spirit of the present specification.

What is claimed is:

1. A cardiac phase selection method, comprising:
obtaining coronary image quality index data of a coronary image dataset, wherein the coronary image quality index data quantifies the medical image quality of coronary medical images of a subject under examination at a plurality of cardiac phases;
selecting a first cardiac phase from the plurality of cardiac phases based on the coronary image quality index data, wherein the first cardiac phase is within a first phase range; and
selecting a second cardiac phase based on the coronary image quality index data, wherein the second cardiac phase is within a second phase range different from the first phase range.

2. The method according to claim 1, wherein the first phase range corresponds to one of systole or diastole, and the second phase range corresponds to the other of systole or diastole.

3. The method according to claim 1, wherein the coronary image quality index data comprises one of an edge strength score, a circularity score, an in-plane score, and a through-plane score of a coronary artery, or is calculated from an edge strength score, a circularity score, an in-plane score, and a through-plane score.

4. The method according to claim 3, wherein the coronary image quality index data is used to quantitatively characterize the image quality of the right coronary artery, the left anterior descending, and the left circumflex branch.

5. The method according to claim 1, wherein a coronary medical image dataset of the subject under examination is obtained by an ECG-less cardiac gating medical imaging device.

6. The method according to claim 1, wherein the coronary image quality index data at the first cardiac phase is a peak or one of a plurality of peaks within the first phase range; and the coronary image quality index data at the second cardiac phase is a peak or one of a plurality of peaks within the second phase range.

7. The method according to claim 6, wherein the selecting a first cardiac phase from the plurality of cardiac phases includes:
selecting a cardiac phase corresponding to the maximum value of the coronary image quality index data from the plurality of cardiac phases as the first cardiac phase.

8. The method according to claim 6, wherein
the first phase range includes phases within a first threshold range on both sides centered on the first cardiac phase; and
the second phase range includes phases within the entire phase range corresponding to the coronary image dataset and outside the first phase range.

9. The method according to claim 8, wherein the first threshold range is the product of an estimated cardiac cycle of the subject under examination and a first ratio, and the first ratio is one of 20% and 25%.

10. The method according to claim 8, further including:
determining a third phase range, wherein the third phase range comprises phases within a second threshold range on both sides centered on the first cardiac phase; and
the second phase is a phase within the third phase range and outside the first phase range.

11. The method according to claim 10, wherein the second threshold range is the product of an estimated cardiac cycle of the subject under examination and a second ratio, and the second ratio is one of 70% and 80%.

12. The method according to claim 1, further including:
obtaining a ventricular volume dataset, wherein the ventricular volume dataset includes ventricular volume data of the subject under examination at a plurality of cardiac phases;
a cardiac phase corresponding to a first peak of the ventricular volume dataset is within the first phase range, and a cardiac phase corresponding to a second peak of the ventricular volume dataset is within the second phase range; and
the first peak is one of a maximum value and a minimum value, and the second peak is the other of the maximum value and the minimum value.

13. A medical imaging method, comprising:
obtaining a coronary image quality score curve of a subject under examination, wherein the coronary image quality score curve is used to quantitatively characterize the medical image quality of a coronary medical image dataset of the subject under examination at a plurality of cardiac phases;
determining a phase corresponding to a peak point in the coronary image quality score curve as a first cardiac phase, wherein a motion amplitude of the heart of the subject under examination at the first cardiac phase is less than a motion amplitude thereof at an adjacent phase, and the first cardiac phase is within one of a systolic range and a diastolic range;
determining a phase corresponding to another peak point in the coronary image quality score curve as a second cardiac phase, wherein a motion amplitude of the heart of the subject under examination at the second cardiac phase is less than a motion amplitude thereof at an adjacent phase, and the second cardiac phase is within the other of the systolic range and the diastolic range; and
obtaining coronary medical images at the first cardiac phase and the second cardiac phase.

14. The method according to claim 13, wherein the coronary medical image dataset of the subject under examination is obtained by an ECG-less cardiac gating medical imaging device.

15. The method according to claim 13, wherein coronary image quality index data includes one of an edge strength score, a circularity score, an in-plane score, and a through-plane score of a coronary artery, or is calculated from an edge strength score, a circularity score, an in-plane score, and a through-plane score.

16. The method according to claim 15, wherein the coronary image quality index data is used to quantitatively characterize the image quality of the right coronary artery, the left anterior descending, and the left circumflex branch.

17. The method according to claim 13, wherein the peak point corresponding to the first cardiac phase is the maximum value of the coronary image quality score curve.

18. The method according to claim 17, wherein:
determining a first phase range, wherein the first cardiac phase is within the first phase range, and the first phase range includes phases within a first threshold range on both sides centered on the first cardiac phase; and
determining a second phase range, wherein the second cardiac phase is within the second phase range, and the second phase range includes phases within the entire phase range corresponding to the coronary image dataset and outside the first phase range.

19. The method according to claim 18, wherein the first threshold range is the product of an estimated cardiac cycle of the subject under examination and a first ratio, and the ratio is one of 20% and 25%.

20. The method according to claim 18, further including:
determining a third phase range, wherein the third phase range comprises phases within a second threshold range on both sides centered on the first cardiac phase; and
the second phase is a phase within the third phase range and outside the first phase range, wherein the second threshold range is the product of an estimated cardiac cycle of the subject under examination and a second ratio, and the second ratio is one of 70% and 80%.
